(19) United States
(12) Patent Application Publication
(10) Pub. No.: US 2006/0041295 A1
(43) Pub. Date: Feb. 23, 2006

(54) Positive fixation percutaneous epidural neurostimulation lead

(76) Inventor: Thomas P. Osypka, Palm Harbor, FL (US)

Correspondence Address: EDWARDS & ANGELL, LLP, P.O. BOX 55874, BOSTON, MA, 02205, US

(21) Appl. No.: 11/201,946

(22) Filed: Aug. 11, 2005

Related U.S. Application Data
(60) Provisional application No. 60/602,191, filed on Aug. 17, 2004.

(51) Int. Cl. A61N 1/05

(52) U.S. Cl. 607/117

(57) ABSTRACT

Disclosed is a lead for percutaneous insertion into an epidural space of a spinal canal, which includes an elongated lead body having opposed proximal and distal end portions. At least one electrode for stimulating a patient is operatively associated with the distal end portion of the lead body. Structure for conducting signals extends through the lead body to connect the electrode to a connecting structure operatively associated with the proximal end portion of the lead body. The connecting structure is capable of engaging a signal generator such that signals can be conducted from a signal generator to the electrode. The distal end portion of the lead body is adapted for movement between a first state, in which the distal end portion has a generally linear configuration, and a second state, in which the distal end portion has an undulating configuration.

### CROSS-REFERENCE TO RELATED APPLICATION

The subject application claims the benefit of commonly-owned, co-pending U.S. Provisional Patent Application Ser. No. 60/602,191, filed on Aug. 17, 2004, the disclosure of which is herein incorporated by reference in its entirety.

### BACKGROUND OF THE INVENTION

1. Field of the Invention

The invention relates to a lead for electrically stimulating a spinal cord and more particularly to an apparatus and method for fixing or otherwise securing such a lead in the epidural space of a spinal column to inhibit lateral lead migration.

2. Background of the Related Art

The basic process by which humans perceive pain begins with the generation of pain signals by nocioreceptors. These pain sensors, which are located throughout the body at the extremities of peripheral nerve fibers, generate pain signals in response to stimuli such as increased pressure, elevated temperature, or chemical alterations. The pain signals generated by the nocioreceptors are transmitted along the peripheral nerve fibers to the spinal cord, from which the peripheral nerve fibers emanate. Once pain signals reach the spinal cord, they propagate along the spinal cord to the brain where the signals are processed and perceived as pain.

The transmission of pain signals is enabled by the multitude of neurons that make up the peripheral nerve fibers and the spinal cord (as well as the brain). Each neuron contains mobile ions that rearrange within the neuron in response to a pain signal to create a potential drop across the neuron. In this way, a pain signal gives rise to an electrical impulse that travels across the neuron. This electrical impulse cannot, however, travel to neighboring neurons, as the neurons making up the nerves and spinal cord are not in electrical contact with one another. Instead, as an electrical impulse representing pain travels across a neuron, the neuron releases a chemical that travels to and reacts with adjacent neurons, causing those neurons to establish the pain-indicating potential drop. In this way, pain signals propagate as an alternating series of electrical impulses (along neurons) and chemical reactions (between neurons).

In many cases, pain results from discrete causes, such as disease, inflammation, or traumatic injury to tissues, which can be identified and treated. This type of pain is referred to as “acute” pain, and is treated by treating the condition causing the pain, with the pain subsiding as the underlying condition is cured. In other cases, pain persists indefinitely (either in a continuous or intermittent manner) despite the completion of the healing process. Such “chronic” pain can happen, for example, when the body is subject to a degenerative condition, such as arthritis, that cannot be healed. Damaged nerves can also cause chronic pain, by generating pain signals even in the absence of a real stimulus or tissue damage. In some rare instances, initially acute pain can become chronic. In any event, chronic pain is associated with a condition that is relatively immune to medical treatment. As such, it is necessary to continually treat the pain independently of any condition that may have given rise to the pain.

One of the most historically common treatments of chronic pain was through medication. As mentioned, the transmission of pain signals to the brain involves a series of alternating electrical impulses and chemical reactions. Medications can be used to disrupt the chemical reactions and “block” pain impulses from reaching the brain. Common medications utilized in blocking pain impulses include morphine and other opioid drugs. However, while such treatment is generally effective in relieving pain, continued use of a morphine-like drug can lead to patient sedation, and has the potential to cause addiction. Further, patients receiving morphine also face the problem of morphine tolerance, meaning that, over time, they require increasingly higher doses of the drug to achieve the same level of pain relief.

Relatively recently, it has been found that establishing an electric field around the spinal cord can serve to effectively reduce or alleviate pain. The electric field interacts with the electrical portion of the pain signal and thereby blocks the transmission of pain impulses along the spinal cord, creating an impaired sensation of the body known as parasthesia. In practice, an electric field is established in the vicinity of the spinal cord by surgically implanting a signal generator and running an electrical lead from the generator to a location adjacent to the spinal cord. This electrical lead is known as a neurological epidural lead. While the implantation of a neurological epidural lead is inappropriate for the temporary treatment required for acute pain due to its invasive nature, the procedure has found use in the continuous treatment of chronic pain.

An example of a typical neurological epidural lead implanted in a spinal canal is shown in FIGS. 1 and 1a, generally labeled 10. Lead 10 has an elongated, substantially linear lead body 12 with opposed proximal 14 and distal 16 end portions, and includes at least two electrodes 18 associated with the distal end portion 16. The lead 10 is located in the epidural space 70 of the spinal canal 71 (the space between the spinal canal wall 72, defined by the ligamentum flavum 73, the vertebrae 74, and the intervertebral discs 76, and the spinal cord 75), such that the electrodes 18 are located in close proximity to the spinal cord 75. The proximal end portion 14 of lead body 12 interfaces with a pulse generator (not shown), such as an implantable pulse generator (IPG) located at a separate location within the body of the patient. Conductive wires (not shown) extend through lead body 12 to operatively connect electrodes 18 to the pulse generator. An electrical potential is applied between pairs of the electrodes 18, and the resulting electric field pervades the spinal column 77 and initiates parasthesia in the patient.

To place the lead 10 in the epidural space 70, a needle is percutaneously inserted through the ligamentum flavum 73. The lead 10 is then passed through the needle and into the epidural space 70, after which the needle is removed. The lead 10 is then manually guided along the spinal canal 71 to the desired location.

While treatment involving the use of the above-described lead has proven somewhat effective, recent studies have indicated that ˜25% of patients who undergo this procedure with initially favorable results experience a subsequent deterioration in therapeutic effectiveness. It is believed that this failure in treatment is caused by post-implantation migration of the electrodes, which, even for movements as small as one millimeter, can cause a significant change in the amount and location of parasthesia induced by lead 10. As such, it is important that the leads remain fixed in place after placement in the epidural space.

To prevent axial movement of the lead 10, a stop 40 (FIG. 1) is placed along lead body 12 outside spinal column 77 near the point where lead 10 passes through ligamentum flavum 73. Stop 40 is sutured to surrounding tissue to prevent lead 10 from moving axially. Transverse movement (i.e., with respect to the long axis of the lead) of the proximal end portion 14 of the lead body 12 is restricted by the surrounding ligamentum flavum 73.

Several methods have been described in the prior art for preventing transverse movement of the distal end portion 16 (FIG. 1) of the lead body 12. First, and most traditionally, the compression of the lead 1 between the spinal cord 75 and the spinal canal wall 72 has been relied upon to secure the lead 10. This tactic, however, has proven unreliable, and it is now believed that excessive lateral migration of the distal end portion of lead occurs fairly regularly in this arrangement.

Others have added a protruding structure to the distal end of the lead body. This protruding structure causes the distal end to anchor into the tissue around the distal end, thus preventing the distal end from moving laterally. Because the distal end cannot move laterally, the lead's electrodes are similarly prevented from moving laterally. An example of this type of lead anchoring system is disclosed in U.S. Pat. No. 5,344,439 to Otten.

However, the lead anchoring systems, such as in Otten, that rely on protruding structures at the distal end of the lead suffer from a drawback related to the physiology of the spinal column. Referring again to FIG. 1, recent research has revealed that the epidural space 70 is not merely the flattened space between the spinal cord 75 and the spinal canal wall 72.¹ Rather, as shown in FIG. 1, the epidural space 70 alternately widens (between vertebra 74, where the spinal canal wall 72 is mainly defined by the ligamentum flavum 73) and narrows (within vertebra 74, where the spinal cord 75 is in substantial contact with the spinal canal wall 72) along the spinal column 77. Consequently, if a lead with a protruding anchoring fixture at the distal end, such as is shown in Otten, was placed in an epidural space such that the distal end was located in a wider portion of the epidural space, there would be insufficient contact between the anchoring protrusion and the spinal canal to prevent lead from moving laterally.
¹Quinn H. Hogan, “Lumbar Epidural Anatomy, A New Look by Cryomicrotome Section,” in Anesthesiology, vol. 75(5), pp. 767-775 (1991).

U.S. Pat. No. 4,538,624 to Tarjan and U.S. Pat. No. 4,549,556 to Tarjan et al., disclose methods of anchoring neurological epidural leads. As disclosed by these patents, an extension extends distally beyond the most distal electrode and terminates in an extension end. The lead is introduced percutaneously into the epidural space through a needle, similar to the process described above. The lead is positioned with the electrodes in the desired location, the extension extending within epidural space distally beyond electrodes. The epidural space is then accessed at a location near extension end, and the extension end is manually retrieved and anchored outside the spinal column. While this procedure results in a securely anchored lead, the process of retrieving and anchoring extension end is difficult and requires an additional puncture to and resulting opening in the spinal canal wall. It is desirable to find a way to anchor distal end 6 easily and without having to puncture the spinal canal wall.

U.S. Pat. No. 5,733,322 to Starkebaum, incorporated herein by reference in its entirety, describes a positive fixation mechanism, including an extension that extends distally beyond the most distal electrode. Implantation is achieved by having the extension placed in a very narrow area of the epidural space. Placement of the extension inside such a narrow area, however, can be very time-consuming and cumbersome.

In all, it is desirable to have a neurological epidural lead that is easily implanted into an epidural space and is adapted to restrict movement of the lead with respect to the spinal cord.

### SUMMARY OF THE INVENTION

The present invention addresses the problems outlined above by providing a novel neurological epidural lead. The novel lead provides a simplified manner for effectively inhibiting lead migration after placement in an epidural space. At the same time, the lead structure allows the lead to be easily directed through the body during lead implantation and placement.

In one embodiment of the subject invention, a lead for percutaneous insertion into an epidural space of a spinal canal has an elongated lead body with opposed proximal and distal end portions. At least one electrode for stimulating a patient is operatively associated with the distal end portion of the lead body. Conductor means for conducting signals extends through the lead body to connect the electrode to connector means operatively associated with the proximal end portion of the lead body. The connector means is capable of engaging a signal generator such that signals can be conducted from a signal generator to the electrode. The distal end portion of the lead body is adapted for movement between a first state, in which the distal end portion has a generally linear configuration, and a second state, in which the distal end portion has an undulating configuration. The generally linear configuration of the first state facilitates passing the lead through a body and into the epidural space and the undulating configuration of the second state causes the distal end portion of the lead body, once situated within the epidural space, to exert outward force on structures defining the spinal canal, thereby affixing the lead within the spinal canal.

In a particular embodiment, at least part of the distal end portion of the lead body is formed of a mechanically elastic material and has an undulating configuration. The lead further comprises a substantially linear stiffening member that selectively extends axially through the distal end portion of the lead body to force the distal end portion of the lead body to assume the generally linear configuration of the first state. The distal end portion of the lead body assumes the undulating unloaded configuration of the second state when the stiffening member is retracted. Preferably, the mechanically elastic material is capable of undergoing a solid-state phase transformation.

The subject invention is also directed to a method for implanting a device for treating pain in a patient. A lead is provided for percutaneous insertion into an epidural space of a spinal canal of the patient. The lead includes an elongated lead body having opposed proximal and distal end portions, wherein the distal end portion of the lead body is adapted for movement between a first state, in which the distal end portion has a generally linear configuration, and a second state, in which the distal end portion has an undulating configuration. A stylet is positioned within the lead body such that the distal end portion of the lead body has the generally linear configuration of the first state. The lead is percutaneously inserted into the epidural space of the patient. The stylet is then retracted such that the distal end portion of the lead body assumes the undulating shape of the second state and contacts structures defining the spinal canal, thereby affixing the lead within the spinal canal.

The subject invention is further directed to a lead for percutaneous insertion into an epidural space of a spinal canal. The lead is capable of interfacing with a signal generator and conducting signals from the signal generator to the spinal canal. The lead includes means for altering the shape of the lead between a first configuration and a second configuration. The first configuration of the lead facilitates insertion of the lead into the epidural space, while the second configuration allows the lead, once situated within the epidural space, to exert outward force on structures of the spinal canal, thereby inhibiting movement of the lead within the spinal canal.

It should be appreciated that the present invention can be implemented and utilized in numerous ways, including without limitation as a process, an apparatus, a system, a device, a method for applications now known and later developed. These and other unique features of the system disclosed herein will become more readily apparent from the following description and the accompanying drawings.

### BRIEF DESCRIPTION OF THE DRAWINGS

So that those having ordinary skill in the art to which the present application appertains will more readily understand how to make and use the same, reference may be had to the drawings wherein:

FIG. 1 is a perspective view of a typical prior art neurological epidural lead implanted in the epidural space of the spinal canal;

FIG. 1a is a side view of a typical prior art neurological epidural lead implanted in the epidural space of the spinal canal, the view taken along line 1a-1a of FIG. 1;

FIG. 2 is a perspective view of a neurological epidural lead constructed in accordance with a preferred embodiment of the present invention, wherein the lead body has a two-dimensional undulating configuration;

FIG. 3 is a side elevational view of the neurological epidural lead of FIG. 2, a portion of the lead body being removed to reveal the conductor coil;

FIG. 4 is an enlarged localized side elevational view in partial cross-section of the neurological epidural lead of FIG. 3, particularly illustrating the lumen defined by the conductor coil;

FIGS. 5a-5c are a series of side elevational views of a neurological epidural lead constructed in accordance with a preferred embodiment of the present invention, the series illustrating the retraction of a stylet from the lead to allow the lead to move from a straight configuration to an undulating configuration;

FIG. 6 is a perspective view of a neurological epidural lead constructed in accordance with a preferred embodiment of the present invention, the lead implanted in the epidural space of the spinal canal and having an undulating configuration that causes the lead to be affixed within the epidural space; and

FIG. 7 is a perspective view of a neurological epidural lead constructed in accordance with another preferred embodiment of the present invention, the lead having a three-dimensional undulating configuration.

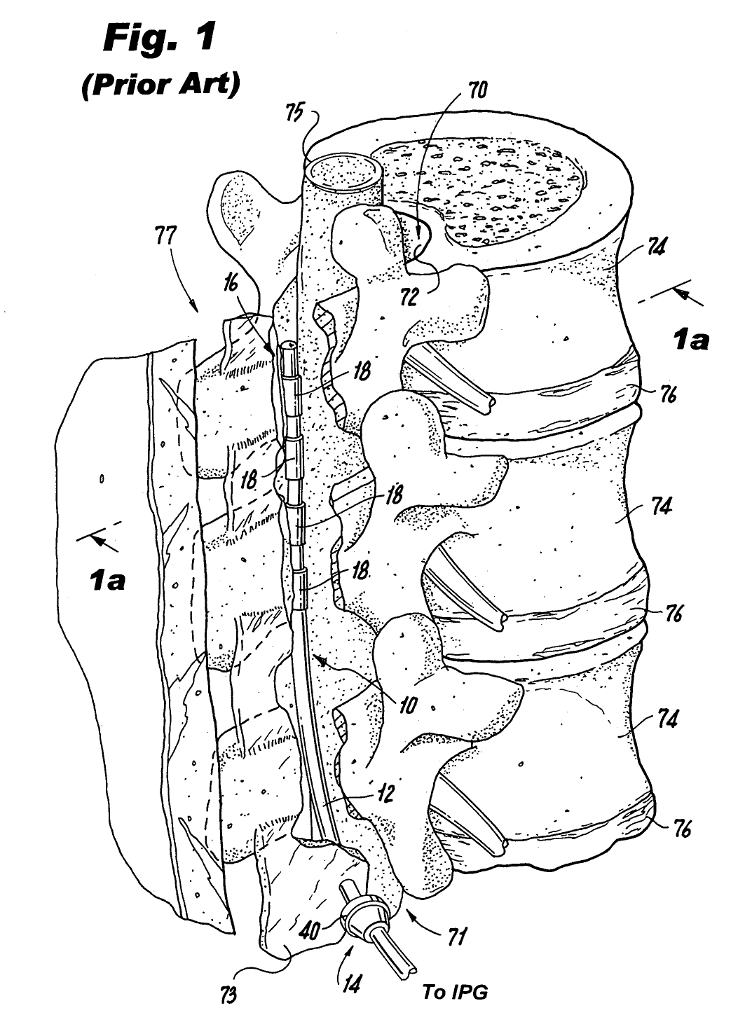

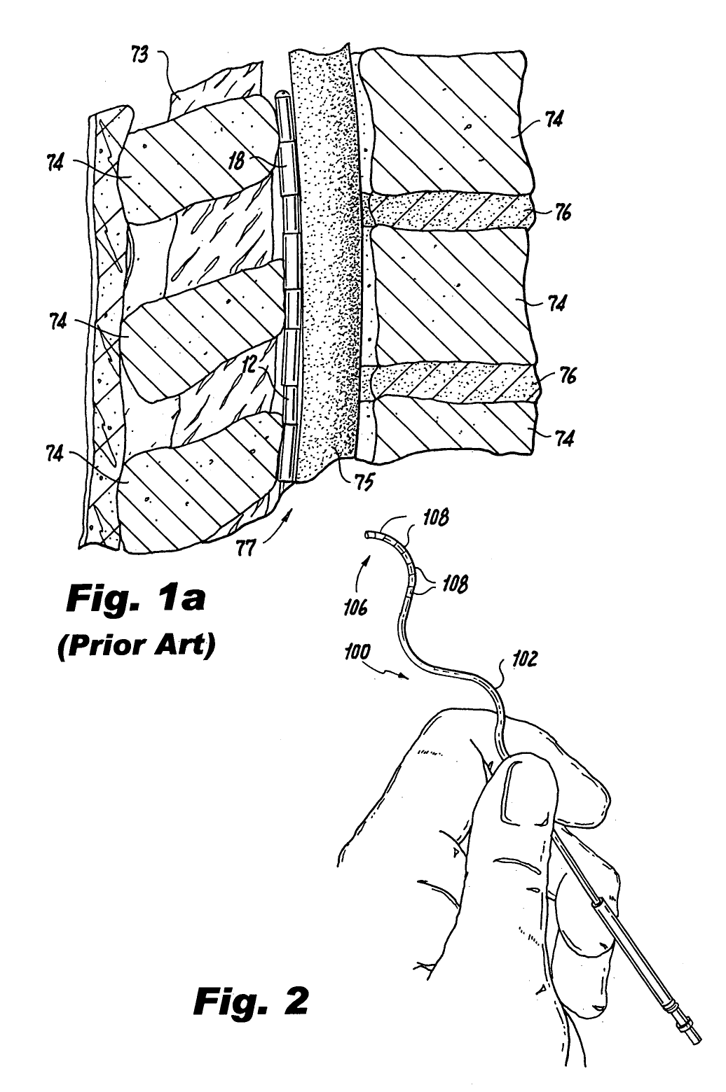

These and other features of the neurological epidural lead of the subject invention will become more readily apparent to those having ordinary skill in the art from the following description of exemplary embodiments.

### DETAILED DESCRIPTION OF PREFERRED EMBODIMENTS

Referring now to the accompanying drawings, wherein like reference numerals identify similar structural features of the present invention, there is illustrated in FIG. 2 a neurological epidural lead 100 constructed in accordance with the present invention. The lead 100 includes an elongated lead body 102 having opposed proximal 104 and distal 106 end portions. Several electrodes 108 are secured to the distal end portion 106 of the lead body 102. Preferably, the lead includes at least two electrodes, although it is possible to utilize a lead with a single electrode. A connector 110 is secured to the proximal end portion 104 of the lead body 102, and is configured to interface with a pulse generator (not shown). The pulse generator could be an implantable pulse generator (IPG) that is implanted within a patient's body, or could be a device that remains external to a patient's body. In a particular embodiment, the connector 110 is a conventional IS-1 type connector, however, those skilled in the art would readily appreciate that other types of connectors could be utilized, such as, for example, IS-4 type connectors, LV-1 type connectors, VS-1 type connectors, and DF-1 type connectors.

At least part of the distal end portion 106 of the lead body 102 is formed of a mechanically elastic material and has an undulating, substantially sinusoidal unloaded configuration. Preferably, the undulating configuration of the distal end portion 106 of the lead body 102 includes the area where the electrodes 108 are secured. The mechanically elastic material is such that the undulating configuration of the distal end portion 106 of the lead body 102 can be substantially straightened by force and will subsequently return to the undulating shape when the force is removed.

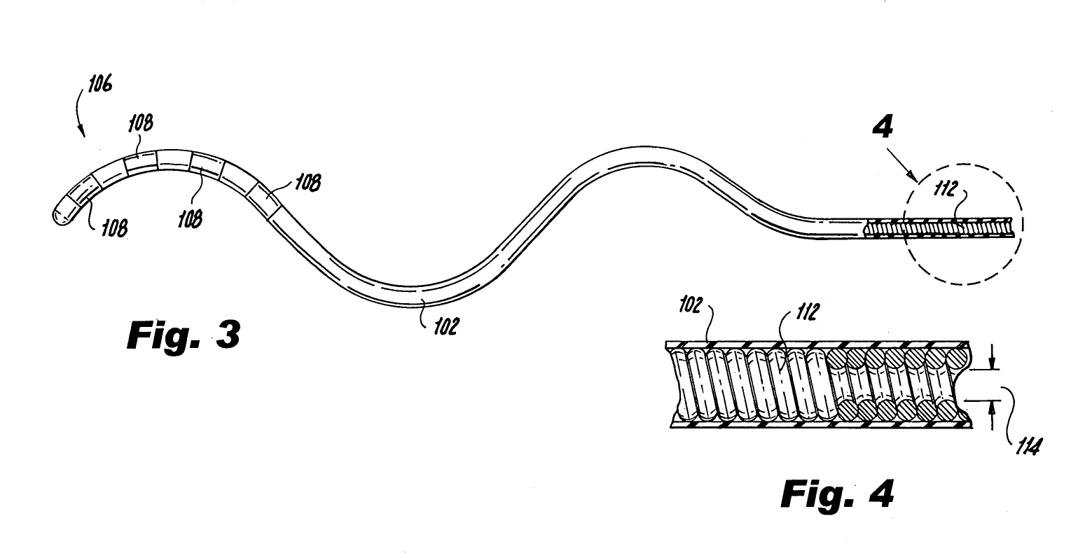

Referring to FIGS. 3 and 4, conductors 112 extend axially through the lead body 102, operatively connecting the connector 110 and the electrodes 108. The conductors 112 are sheathed in insulating material and arranged in a coil. The coil may consist of a single conductor or may be a multi-filar coil. An example of a suitable multi-filar coil assembly is disclosed in U.S. Patent Application No. 2003/0092303 to Osypka, the disclosure of which is herein incorporated by reference in its entirety. Preferably, respective conductors 112 connect each electrode 108 to connector 110, although it is also possible to connect multiple electrodes with a single conductor. Through the engagement of the connector 110 by an IPG, the conductors 112 allow signals to pass from the IPG to the electrodes 108. The conductors 112, so arranged, define a lumen 114.

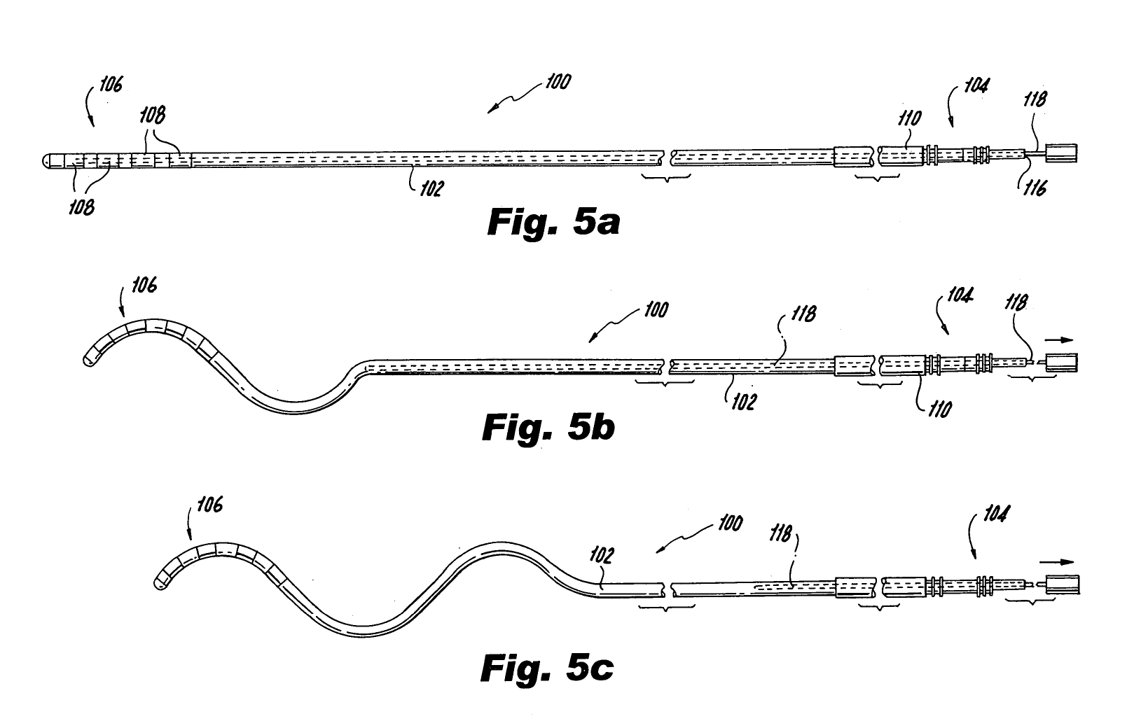

Referring to FIGS. 5a-c, a port 116 allows a stylet 118 to be selectively inserted into and retracted from the lead body 102. The inserted stylet 118 extends through the lumen 114 defined by the multi-filar coil arrangement of the conductors 112. The stylet 118 is of sufficient stiffness with respect to the lead body 102 so as to force the distal end portion 106 of the lead body 102 into a substantially straight configuration when inserted through the lead body 102 and into the distal end portion 106. However, when the stylet 118 is retracted from the distal end portion 106, the mechanically elastic material composing the undulating part of the distal end portion 106 causes the lead 100 to resume the undulating configuration. The selective insertion and retraction of stylet 118 allows the distal end portion 106 to be selectively moved between a substantially linear configuration (shown in FIG. 5a) and the sinusoidal unloaded configuration (represented in FIG. 5c).

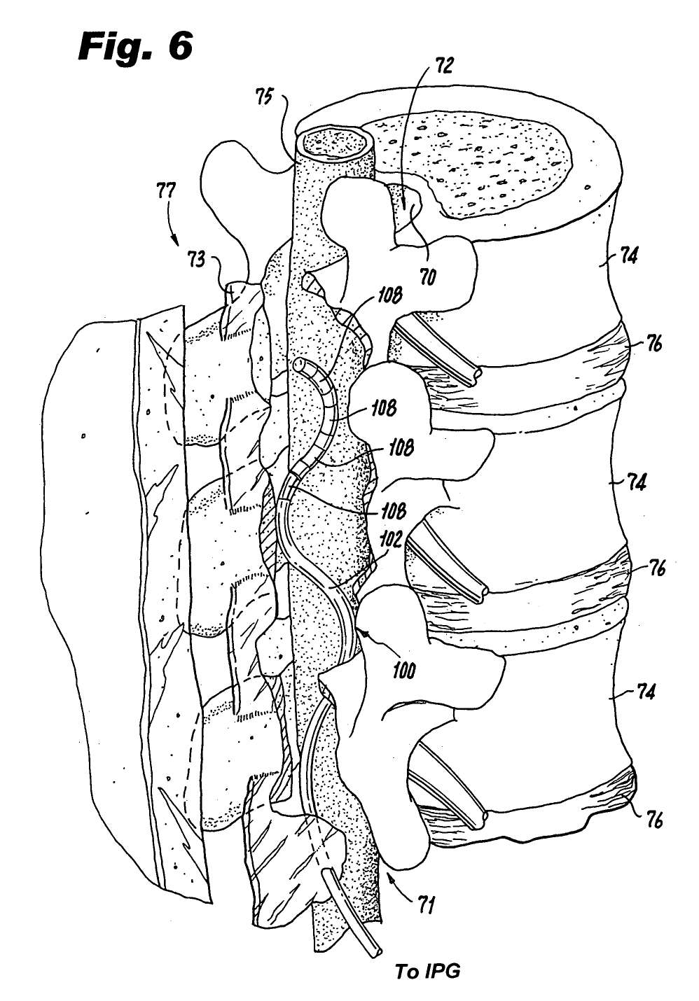

Referring to FIGS. 5a-5c and 6, in use, lead 100, with the stylet 118 occupying the distal end portion 106 of the lead body 102, is inserted percutaneously into the body. With the stylet 118 so inserted, the distal end portion 106 is substantially straight, facilitating navigation of the lead 100 through the body, and, specifically, through the narrow regions of the epidural space 70. Lead 100 is moved through the body and positioned appropriately in epidural space 70 to allow the electrodes 108 to establish the desired electric field around the spinal cord 75.

After the lead 100 is properly positioned, the stylet 118 is withdrawn and distal end portion 106 attempts to assume the undulating shape. However, the undulating configuration is dimensioned to allow the distal end portion 106 of the lead body 102 to contact and exert outward force on the surrounding spinal cord 75 and/or spinal canal wall 72 before reaching the unloaded undulating configuration, thereby stabilizing the position of the lead 100 within the epidural space 70 and preventing lateral migration of the lead 100. Further, in attempting to assume the undulating configuration, the electrodes 108 in the distal end portion 106 are pressed against the spinal cord 75, thereby improving the electrical stimulation. Finally, after the lead 100 has been secured in the epidural space 70, the connector 110 is connected to an IPG (not shown) to complete the procedure.

In the preferred embodiment of FIGS. 5a-5c, the stylet 118 extends the length of the lead 100 in occupying the distal end portion 106 of lead body 102, and is fully retracted from the lead 100 after lead placement. However, another preferred embodiment utilizes a stylet (or other stiffener) that is shorter than the lead and occupies only a distal end portion of the lead body. A flexible guide wire extends from the shorter stylet to an opening in the connector, allowing external manipulation of the stylet via the guide wire. Such a structure allows the lead to move from a straight to an undulating configuration either by partial retraction of the shorter stylet, with the stylet remaining within the lead but not within the distal end portion, or by full retraction. In still another preferred embodiment, the lead body is sufficiently compliant to allow a guide wire alone to act to straighten the lead upon insertion, thereby obviating altogether the need for a stylet. In yet another preferred embodiment, a telescoping stiffener is included in the lead, such that the stiffener may be collapsed to allow the lead to assume an undulating configuration without removing the stiffener from the lead.

In a preferred embodiment, the mechanically elastic material composing the undulating part of the distal end portion 106 of the lead body 102 undergoes a solid-state phase change when moving between the undulating configuration and the generally linear configuration. Such a phase change is often accompanied by a shape change in the material, this shape change serving to enhance the magnitude of elastically recoverable deformation, as is well known to those skilled in the art. Materials capable of undergoing such a solid-state phase change are commonly referred to as shape memory materials, some examples being nickel-titanium alloy, copper-zinc-aluminum alloy, and copper-aluminum-nickel alloy. The use of a shape memory material in the distal end portion 106 of the lead body 102 thereby increases the amount of shape change that can be achieved in the lead 100 when moving between the straight and undulating configurations.

In another preferred embodiment, a solid-state phase change is induced not by mechanical deformation, as described above, but through temperature change. The distal end portion 106 of the lead body 102 is formed, at least in part, of a material having multiple stable solid phases below the melting temperature, the transition from one phase to another requiring only limited diffusion (so-called “diffusionless” phase changes). A temperature change prompts the material to change phases, such phase change (as with the above-described mechanically-induced case) being accompanied by a shape change. In a particular embodiment, a lead can be moved between an undulating and a substantially straight configuration entirely through thermally induced shape change, removing the need for a stylet. In still another preferred embodiment, the material composing at least part of the distal end portion 106 is a piezoelectric material, such as quartz, rather than a phase changing material. In that case, shape change in the distal end portion 106 is induced, at least in part, by the establishment of an electric field, which causes the material to change shape.

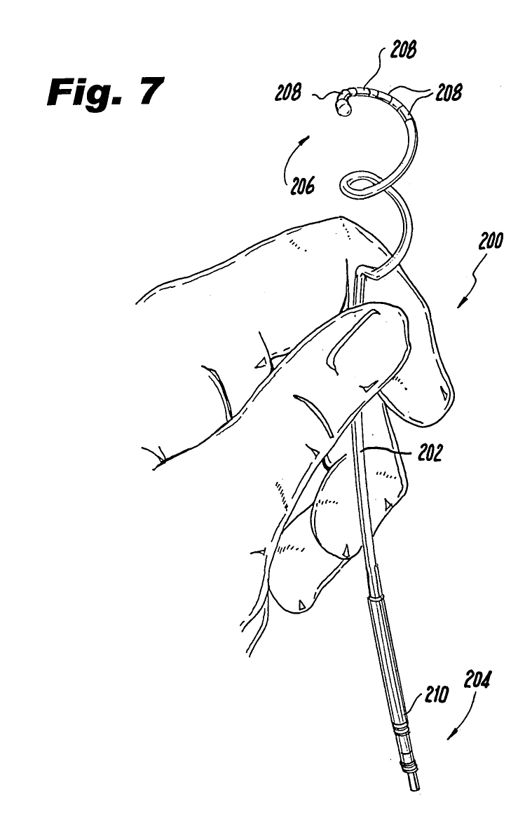

Referring to FIG. 7, in another preferred embodiment of the present invention, lead 200 includes an elongated lead body 202 with opposed proximal 204 and distal 206 end portions. The distal end portion 206 has a substantially helical unloaded configuration extending in three dimensions. A connector 210 is secured to the proximal end portion 204 of the lead body 202, and is configured to interface with a pulse generator (not shown). For some applications and/or physiologies, use of such a three-dimensional configuration for distal end portion 206 is advantageous, allowing for more secure fixation of the lead 200 within a body and/or delivering better therapeutic performance. Along these lines, the present invention is not specifically limited to specific unloaded shapes of the distal end portion, but contemplates any number of two-dimensional and three-dimensional shapes as may be desired for a particular application, whether these shapes involve regular, repeating patterns or irregular configurations. Further, leads can be designed with shapes specifically suited for the particular anatomy of the patient.

It should also be understood that the foregoing is only illustrative of exemplary and preferred embodiments, as well as principles of the subject invention. Those skilled in the art will readily appreciate that various modifications can be made without departing from the scope and spirit of the invention, as demonstrated below.

The present invention contemplates a variety of possible arrangements for the conductors in an implantable lead. For example, in another preferred embodiment, the conductors can be replaced by low resistance stranded wires or cables, or by drawn filled tubing (DFT). In a particular embodiment, such DFT extends through multi-lumen tubing in order to connect the connector and the electrodes. An example of such multi-lumen tubing is disclosed in U.S. Patent Application No. 60/622,864 to Osypka, the disclosure of which is herein incorporated by reference in its entirety. Preferably, one of the lumens is left available for receiving a stylet or other stiffening member, which is selectively inserted to effectuate the straightening of the lead. Alternatively, such DFT wires may each be encased in respective insulation tubes.

In other preferred embodiments, the conductors of the lead serve both to determine the unloaded shape of the lead and to provide the ability for the lead to recover this unloaded shape following deformation. The lead body is then formed of flexible materials such that the lead body generally conforms to the shape of the conductors. For example, the conductors can be arranged in a multi-filar coil and the coil initially deformed into an undulating configuration. The initial deformation can be plastic, such that strain hardening of the conductor material allows subsequent deformations of the coil between the undulating configuration and a forcibly straightened configuration to occur elastically. Alternatively, the coil can be deformed elastically and annealed while maintained in this deformed state, such that the undulating configuration remains after unloading. In a particular embodiment, the conductors are formed of a shape memory material (either mechanical, thermal, or both) that determines or enhances the range of elastic deformation of the conductors.

While the invention has been described with respect to preferred embodiments, those skilled in the art will readily appreciate that various changes and/or modifications can be made to the invention without departing from the spirit or scope of the invention as defined by the appended claims.

What is claimed is:

1. A lead for percutaneous insertion into an epidural space of a spinal canal, the lead comprising:
a) an elongated lead body having opposed proximal and distal end portions, wherein the distal end portion is adapted for movement between a first state in which the distal end portion has a generally linear configuration and a second state in which the distal end portion has an undulating configuration;
b) at least one electrode operatively associated with the distal end portion of the lead body for stimulating a patient;
c) connector means operatively associated with the proximal end portion of the lead body for connecting to a signal generator; and
d) conductor means extending through the lead body for conducting signals between the at least one electrode and the connector means, and whereby the generally linear configuration of the first state facilitates insertion of the lead into the epidural space and the undulating configuration of the second state allows the distal end portion of the lead body, once situated within the epidural space, to exert outward force on structures of the spinal canal, thereby affixing the lead within the spinal canal.

2. A lead as recited in claim 1, wherein the undulating configuration of the second state is generally two-dimensional.

3. A lead as recited in claim 2, wherein the two-dimensional undulating configuration is substantially sinusoidal.

4. A lead as recited in claim 1, wherein the undulating configuration of the second state is generally three-dimensional.

5. A lead as recited in claim 4, wherein the three-dimensional undulating configuration is substantially helical.

6. An implantable lead as recited in claim 1, wherein at least part of the distal end portion of the lead body is formed of a mechanically elastic material and has an undulating configuration and is capable of moving elastically between the undulating configuration and a substantially linear configuration.

7. An implantable lead as recited in claim 6, wherein the lead further comprises a substantially linear stiffening member that selectively extends axially through the distal end portion of the lead body to force the distal end portion of the lead body to assume the generally linear configuration of the first state, the distal end portion of the lead body assuming the undulating configuration of the second state when the stiffening member is retracted.

8. A lead as recited in claim 7, wherein the substantially linear stiffening member is one of a guide wire and a stylet.

9. A lead as recited in claim 6, wherein the mechanically elastic material forming at least part of the distal end portion of the lead body undergoes a solid state phase change when moving between the undulating configuration of the second state and the generally linear configuration of the first state.

10. A lead as recited in claim 9, wherein the mechanically elastic material is a metal alloy selected from the group consisting of nickel-titanium alloy, copper-zinc-aluminum alloy, and copper-aluminum-nickel alloy.

11. An implantable lead as recited in claim 1, wherein the conductor means defines at least in part means for facilitating movement of the distal end portion of the lead body between the first and second states.

12. An implantable lead as recited in claim 11, wherein the lead body is flexible and the conductor means is at least partially formed of a mechanically elastic material and has an undulating configuration and is capable of moving elastically between the undulating configuration and a substantially linear configuration.

13. A lead as recited in claim 12, wherein the mechanically elastic material forming at least part of the conductor means undergoes a solid state phase change when moving between the undulating configuration of the second state and the generally linear configuration of the first state.

14. A lead as recited in claim 13, wherein the mechanically elastic material is a metal alloy selected from the group consisting of nickel-titanium alloy, copper-zinc-aluminum alloy, and copper-aluminum-nickel alloy.

15. An implantable lead as recited in claim 1, wherein the conductor means includes a multi-filar coil of helically wrapped conductors.

16. An implantable lead as recited in claim 1, wherein the conductor means includes a plurality of low resistance stranded cables.

17. A method for implanting a device for treating pain in a patient comprising the steps of:
a) providing a lead for percutaneous insertion into an epidural space of a spinal canal of the patient, the lead comprising an elongated lead body having opposed proximal and distal end portions, wherein the distal end portion is adapted for movement between a first state in which the distal end portion has a generally linear configuration and a second state in which the distal end portion has an undulating configuration;
b) positioning a stylet within the lead body such that the distal end portion of the lead body has the generally linear configuration of the first state;
c) percutaneously inserting the lead into the epidural space of the patient; and
d) retracting the stylet such that the distal end portion of the lead body assumes the undulating shape of the second state and contacts structures defining the spinal canal, thereby affixing the lead within the spinal canal.

18. A lead for percutaneous insertion into an epidural space of a spinal canal, the lead being capable of interfacing with a signal generator and conducting signals from the signal generator to the spinal canal and comprising:
means for altering the shape of the lead between a first configuration that facilitates insertion of the lead into the epidural space and a second configuration that allows the lead, once situated within the epidural space, to exert outward force on structures of the spinal canal, thereby inhibiting movement of the lead within the spinal canal.

19. A lead as recited in claim 18, wherein the second configuration of the lead is an undulating configuration.

20. A lead as recited in claim 18, wherein the first configuration of the lead is a substantially linear configuration.
